(19) United States
(12) Patent Application Publication
(10) Pub. No.: US 2025/0375665 A1
(43) Pub. Date: Dec. 11, 2025

(54) SYSTEMS AND METHODS FOR PREDICTING AND OPTIMIZING PHYSIOLOGICAL PERFORMANCE

(71) Applicant: Government of the United States as represented by the Secretary of the Air Force, Wright-Patterson AFB, OH (US)

(72) Inventors: Joshua Hagen, Cincinnati, OH (US); Gurtej S. Grewal, Beavercreek, OH (US)

(21) Appl. No.: 19/308,712

(22) Filed: Aug. 25, 2025

Related U.S. Application Data
(63) Continuation of application No. 17/400,565, filed on Aug. 12, 2021.
Application No. 17/400,565 is a continuation of application No. 16/105,569, filed on Aug. 20, 2018, now abandoned.
(60) Provisional application No. 62/547,449, filed on Aug. 18, 2017.

(51) Int. Cl. A63B 24/00; G06F 1/16; G06F 16/903; G06F 17/18; G06Q 10/04; G16H 20/30; G16H 20/70

(52) U.S. Cl. CPC A63B 24/0062; G06F 1/163; G06F 16/90335; G06F 17/18; G06Q 10/04; G16H 20/30; G16H 20/70; A63B 2024/0065; A63B 2024/0068; A63B 2230/065

(57) ABSTRACT

Systems and methods for predicting/optimizing physical performance including: receiving a selection of one or more individual performance parameters for a future instance of an event, the individual performance parameters indicative of a desired physical performance; receiving a selection of one or more team performance parameters, the team performance parameters indicative of a physical performance of a team; collecting data related to the individual performance parameters and the team performance parameters from past occurrences of the particular event; identifying a subset of training parameters, from within a collection of training parameter data associated with past occurrences; and providing a training program.

### CROSS REFERENCE TO RELATED APPLICATION

This application is a continuation of U.S. application Ser. No. 17/400,565 filed Aug. 12, 2021, which claims the benefit of U.S. application Ser. No. 16/105,569, filed Aug. 20, 2018 (pending), which claims the benefit of and priority to co-pending U.S. Provisional Application Ser. No. 62/547,449 filed Aug. 18, 2017. The contents of these applications are incorporated herein by reference in their entirety.

### GOVERNMENT INTEREST

The invention described herein may be manufactured and used by or for the Government of the United States for all governmental purposes without the payment of any royalty.

### FIELD OF THE INVENTION

The present invention relates generally to assessing individual and team physiology, physical activity, cognitive activity, and so forth. More particularly, the present invention relates to correlating individual and team physiology, physical activity, cognitive activity, and so forth to performance.

### BACKGROUND OF THE INVENTION

Countless physiological and activity (e.g., body movement) related parameters can now be measured non-invasively using various commercially-available wearable sensor and related software technologies including, but not limited to, products under the trade names Fitbit®, Apple Watch®, Zebra Motionworks®, Zephyr™ Performance Systems, Omegawave®, etc., Examples of the physiological parameters that are often tested include heart rate, heart rate variability (sympathetic and parasympathetic), respiration rate, blood oximetry, etc. Activity-related parameters include change of direction, acceleration, distance ran/walked, speed, explosiveness, etc. Additional parameters may be derived from physiological parameters, activity-related parameters, or combinations thereof with personal data regarding the user. For example, mostly all commercially-available fitness tracking products estimate caloric burn using height, weight, a level physical activity detected, and physiological parameters (such as a heart rate during the physical activity). Similarly, Omegawave® includes a “CNS” (central nervous system) score that uses “DC-Potential measurement to monitor and manage signs of fatigue in your Central Nervous System.” The Zephyr™ system uses “physiological load” (defined generally as a cumulative index of effort based on heart rate over a period of time), “physiological intensity” (defined generally as an instantaneous index of effort based on heart rate at that moment), “mechanical load” (defined generally as a cumulative index of effort based on acceleration over a period of time), “mechanical intensity” (defined generally as an instantaneous index of effort based on acceleration at a particular moment), “training load” (an average of the mechanical and physiological load), and “training intensity” (average of the mechanical and physiological intensity), to name a few.

While these commercially-available products are good at collecting and tracking many types of data for a particular individual, there remains a need for improvement as to how this data is used to predict and/or optimize performance. For example, the current commercially-available products are typically focused on displaying data being collected to give immediate feedback during training. In other words, the conventional products are typically focused on providing a “snap shot” of a particular individual's health at a particular moment in time without providing information that could be used to optimize the individual's future performance (over the next days or weeks, for example).

Another deficiency of conventional fitness tracking devices is that the comparing of each particular individual's data to so-called “normal ranges” that are based solely or primarily on physical and measurable information provided by the particular individual to the device (for example, age, height, weight). However, analysis of the data collected is quite user-dependent and “normal ranges” often specific to the particular individual. While certain evaluations are conventionally used to determine an individual's performance (such as physical battery tests, cognitive scores, blood biomarkers, heart rate, heart rate variability, autonomous and central nervous system responses, etc.), the results from each test is highly variable by individual. As a result, data obtained from these tests, when compared against normal ranges generalized to a large population of similar age, height, and weight, will not provide a complete picture for the particular individual.

Another difficulty with these conventional devices is the abundance of data, which complicates the sorting and identifying of which parameters are most important or significant for the particular individual. Moreover, parameters are activity dependent. That is, a parameter may be important for one type of sport or activity but not for a different sport or activity. What is needed therefore is a system for identifying parameters importance for a particular individual based on the sport or activity the individual is competing. Further, there is a need for methods of using the data collected for these important parameters to predict and optimize future performance for that same type of sport or activity.

Another issue with commercially-available fitness tracking products is that when a measured parameter is out of the normal range (e.g., heart rate is higher than normal), the devices does not providing information on how to bring the parameter back into the normal range apart from changing the current activity being performed (e.g., heart rate is high, so take a rest or lower activity level to lower the heart rate). Therefore, it would be further advantageous for the system to not only identify the important or significant parameters for a particular sport or activity, but to also identify certain manipulatable parameters (those parameters that may be changed directly or indirectly) that are best suited to keep the particular individual within an optimal range.

Still another deficiency of commercially-available fitness tracking products with respect to performance is that the data collected focuses on the particular individual's health without considering how the particular individual's health relates to a group or team. While it may be easy to adjust an individual's training schedule for individual activities (such as sports including tennis, running, golf, boxing, etc.), adjusting the training schedules for team sports are more difficult because the schedules are designed around the entire team being able to participate. For example, a football team often implements new offensive plays and formations in the week leading up to a particular game based on the particular style of defense of the upcoming opponent. Conventionally, the plays are practiced to “game speed” with the team's offensive competitors so as to simulate the upcoming game and better prepare the offense as a whole. On the other hand, if certain competitors are not performing optimally (are injured or sore from a previous game), then the team's offensive performance may be dictated more by a degree of soreness on game day as compared to the activity level of practice. In such instances, it may be advantageous to lower the practice speed or level to permit the team's offense to be rested for optimal performance on game day.

As a result, there remains a need for a system that may identify important or significant parameters based on a particular sport or activity, for individuals, teams, or both, using data collected from individuals of the team to design training schedules that optimizes performance of the individual, team, or both.

### SUMMARY OF THE INVENTION

The present invention overcomes the foregoing problems and other shortcomings, drawbacks, and challenges of conventional fitness tracking products with respect to optimizing individual performance, team performance, or both. While the invention will be described in connection with certain embodiments, it will be understood that the invention is not limited to these embodiments. To the contrary, this invention includes all alternatives, modifications, and equivalents as may be included within the spirit and scope of the present invention.

According to one embodiment of the present invention, the above and other needs are met by a method for predicting physical performance for a future event comprising (a) selecting one or more performance parameters for the future event indicative of physical performance; (b) collecting data for the one or more performance parameters selected in step (a) from a plurality of past events that are substantially similar to the future event; (c) collecting data for a plurality of training parameters from prior to each of the plurality of past events; (d) for each of the one or more performance parameters selected in step (a), comparing the collected data for the plurality of training parameters of step (c) to the collected data for the performance parameter of step (b) for each of the plurality of past events to determine which of the plurality of training parameters are statistically significant training parameters; (e) determining an optimal performance range for one or more of the statistically significant training parameters; (f) collecting data for the one or more statistically significant training parameters having an optimal performance range as determined in step (e) prior to the future event; and (g) for each of the one or more statistically significant training parameters having an optimal performance range determined in step (e), predicting performance for the future event by comparing the collected data in step (f) with the optimal performance range determined in step (e).

According to certain embodiments, the method further includes optimizing performance for the future event by (h) determining which of the statistically significant training parameters are able to be manipulated; and (i) providing a training program for manipulating one or more of the statistically significant training parameters determined to be manipulatable in step (h) to conform to the optimal performance range of the readiness index developed in step (e) prior to the future event. In certain embodiments, step (h) includes determining whether at least one of the statistically significant training parameters is able to be manipulated indirectly by comparing the collected data of step (c) for the statistically significant training parameter with the collected data of step (c) for at least one of the other training parameters to determine whether the other training parameter is a statistically significant training variable for the statistically significant training parameter.

According to some embodiments, the one or more performance parameters selected in step (a) include one or more team-based metrics, step (b) includes collecting data for the one or more team-based metrics, and step (c) includes collecting individual-based training parameter data for each member of the team and converting the collected data for each of the training parameters to arrive at team-based training parameter data for comparison with the collected data for the team-based metrics of step (b). In certain embodiments, the converted team-based training parameter data of step (c) for each of the training parameters is compared in step (d) to the collected data for the team-based metrics of step (b) to determine which of the training parameters are statistically significant to the team-based metrics selected in step (a).

According to some embodiments, the one or more performance parameters selected in step (a) include one or more individual-based performance parameters, step (b) includes collecting data for the individual-based performance parameters, and step (c) includes collecting individual-based training parameter for comparison in step (d) with the collected data for the individual-based performance parameter data of step (b).

According to some embodiments, the data collected in step (c) is grouped into a plurality of time periods based on how long the data was collected prior to the corresponding past event and step (d) includes determining the statistically significant training parameters for each of the plurality of time periods.

According to some embodiments, the plurality of past events are performed by the same individual or team of individuals as the future event. In some embodiments, the plurality of past events are assigned a plurality of characteristics indicative of the past event and saved to a database, the method further comprising selecting the plurality of past events for comparison of the collected data in step (d) by matching expected characteristics of the future event to one or more of the characteristics assigned to the plurality of past events.

According to another embodiment of the present invention, a method for optimizing physical performance for a future event includes (a) selecting one or more performance parameters for the future event indicative of physical performance; (b) collecting data for the one or more performance parameters selected in step (a) from a plurality of past events that are substantially similar to the future event; (c) collecting a plurality of heart rate variability values from prior to each of the plurality of past events; (d) collecting data for one or more training parameters from prior to each of the plurality of past events; (e) determining an optimal performance range for heart rate variability prior to the future event by comparing the collected data for the plurality of heart rate variability values of step (c) to the collected data for the one or more performance parameters of step (b) for each of the plurality of past events; (f) determining which of the one or more training parameters are statistically significant to heart rate variability by comparing the collected data for the one or more training parameters of step (d) to the collected data from the plurality of heart rate variability values of step (c) for each of the past events; and (g) providing a training program for manipulating heart rate variability to conform to the optimal performance range determined in step (e) by manipulating one or more of the training parameters determined to be statistically significant in step (f) prior to the future event to optimize performance during the future event.

According to certain embodiments, the data collected in step (c) is grouped into a plurality of time periods based on how long the data was collected prior to the corresponding past event and step (e) includes determining the optical performance range for heart rate variability for each of the plurality of time periods.

According to certain embodiments, the plurality of past events are performed by the same individual or team of individuals as the future event. According to some embodiments, the plurality of past events are assigned a plurality of characteristics indicative of the past event and saved to a database, the method further comprising selecting the plurality of past events for comparison of the collected data in step (e) by matching expected characteristics of the future event to one or more of the characteristics assigned to the plurality of past events.

According to yet another embodiment of the present invention, a system for optimizing physical performance for a future event includes a user interface configured for selecting one or more performance parameters for the future event indicative of physical performance, selecting a plurality of training parameters that are potentially indicative of physical performance for the future event, uploading collected data for the selected performance parameters from a plurality of past events that are substantially similar to the future event, and uploading collected data for the selected training parameters from prior to each of the plurality of past events. The system further includes a computing program configured to compare the collected data for the selected training parameters to the collected data for the selected performance parameters to determine which of the selected training parameters are statistically significant training parameters for each of the selected performance parameters, determine which of the training parameters are able to be directly manipulated, comparing the collected data for the statistically significant training parameter from the plurality of past events with the collected data for the training parameters able to be directly manipulated to determine which of the directly manipulatable training parameters are a statistically significant training variable for the at least one statistically significant training parameter that is unable to be directly manipulated, and provide a training program for indirectly manipulating the statistically significant training parameter that is unable to be directly manipulated by directly manipulating one or more of the statistically significant training variables.

According to certain embodiments, the computing program is further operable to determine an optimal performance range for the at least one statistically significant training parameter that is determined to be unable to be directly manipulated, wherein the training program includes manipulating one or more of the statistically significant training variables such that the statistically significant training parameter conforms to the optimal performance range prior to the future event.

According to some embodiments, the data collected for the selected training parameters is grouped into a plurality of time periods based on how long the data was collected prior to the corresponding past event and the computing program is further operable to determine which of the selected training parameters are statistically significant training parameters for each of the plurality of time periods.

According to some embodiments, they system further includes a database for storing the collected data of the plurality of past events based on a plurality of characteristics indicative of the past event, the user interface further configured for selecting the plurality of past events for comparison of the collected data by matching expected characteristics of the future event to one or more of the characteristics of the plurality of past events.

According to certain embodiments, the system further includes one or more wearable fitness monitors for collecting data for one or more of the selected training parameters.

According to certain embodiments, the one or more performance parameters includes one or more team-based metrics and the plurality of training parameters include individual-based training parameters, the computing program further configured to convert the collected data for each of the individual-based training parameters to arrive at team-based training parameter data for comparison with collected data for the team-based metrics to determine the statistically significant training parameters for a team.

According to certain embodiments, the one or more performance parameters includes one or more individual-based performance parameters and the plurality of training parameters include individual-based training parameters for determining statistically significant training parameters for the individual by comparing collected data for the individual-based training parameters with collected data for the individual based performance parameters.

According to another embodiment of the present disclosure, a system for optimizing human physiological performance with respect to a set of physiological and activity-related parameters related to a particular event includes a display configured to operate as a user interface capable of receiving a user input and displaying an output to the user, a memory, storing one or more processor-readable instructions, and a processor, capable of receiving user inputs and displaying user outputs on the display based on the one or more processor-readable instructions. The instructions are configured to cause the system to: receive a selection of one or more individual performance parameters for a future instance of the particular event from the user interface, the individual performance parameters indicative of a desired physical performance of an individual for the particular event; receive a selection of one or more team performance parameters for a future instance of the particular event from the user interface, the team performance parameters indicative of a physical performance of a team for the particular event; collect data related to the individual performance parameters and the team performance parameters from past occurrences of the particular event; identify a subset of training parameters, from within a collection of training parameter data associated with past occurrences of the particular event or related events, that exhibit a statistically significant correlation with the selected individual performance parameters and the selected team performance parameters; and provide a training program, accessible on the user interface, listing each of the optimal training parameters and recommending a training routine for optimizing each of the individual performance parameters and the team performance parameters.

According to another embodiment of the present disclosure, a method for optimizing human physiological performance with respect to a set of physiological and activity-related parameters related to a particular event, the method comprising: receiving, via a user interface, a selection of one or more individual performance parameters for an instance of the particular event, the individual performance parameters indicative of a desired physical performance of an individual for the particular event; receiving, via the user interface, a selection of one or more team performance parameters for an instance of the particular event, the team performance parameters indicative of a physical performance of a team for the particular event; collecting data related to the individual performance parameters and the team performance parameters from past occurrences of the particular event; accessing training parameter data associated with the past occurrences of the particular event; identifying, from the training parameter data, a subset of training parameters that exhibit a statistically significant correlation with the individual performance parameters and the team performance parameters; generating a training program that lists each of the identified training parameters and recommends a training routine for optimizing the individual performance parameters and the team performance parameters; and providing the training program to a user via the user interface.

According to yet another embodiment of the present disclosure, a system for facilitating improvement of human physiological performance relative to a particular event, the system comprising: a display configured as a user interface for receiving user input and presenting information to a user; a memory storing one or more processor-executable instructions; and a processor operably coupled to the display and the memory. The processor is configured to execute the instructions to: obtain, via the user interface, input identifying one or more individual performance parameters associated with a future instance of the particular event, the individual performance parameters indicative of a desired outcome for an individual participant in the event; obtain, via the user interface, input identifying one or more team performance parameters associated with the future instance of the particular event, the team performance parameters indicative of a desired outcome for a group of participants in the event; access stored data representing historical instances of the particular event or related events, the historical data comprising training parameter data and corresponding performance outcomes; determine a set of training parameters, from the historical training parameter data, that exhibit a predictive relationship with the identified individual performance parameters and team performance parameters, based on a statistical analysis of the historical data; generate and display, via the user interface, a training program including training recommendations corresponding to the determined training parameters, the training recommendations being configured to guide the individual in achieving the identified individual performance parameters and team performance parameters.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a flowchart illustrating a process for predicting and optimizing performance according to one embodiment of the present invention.

FIG. 2 is a flowchart illustrating a process for predicting and optimizing performance according to another embodiment of the present invention.

FIG. 3 is a schematic illustration of a computer system suitable for use with embodiments of the present invention.

FIGS. 4-8 are charts and graphs illustrating findings from an exemplary comparison of training parameter data to performance parameter data for events for a particular team.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

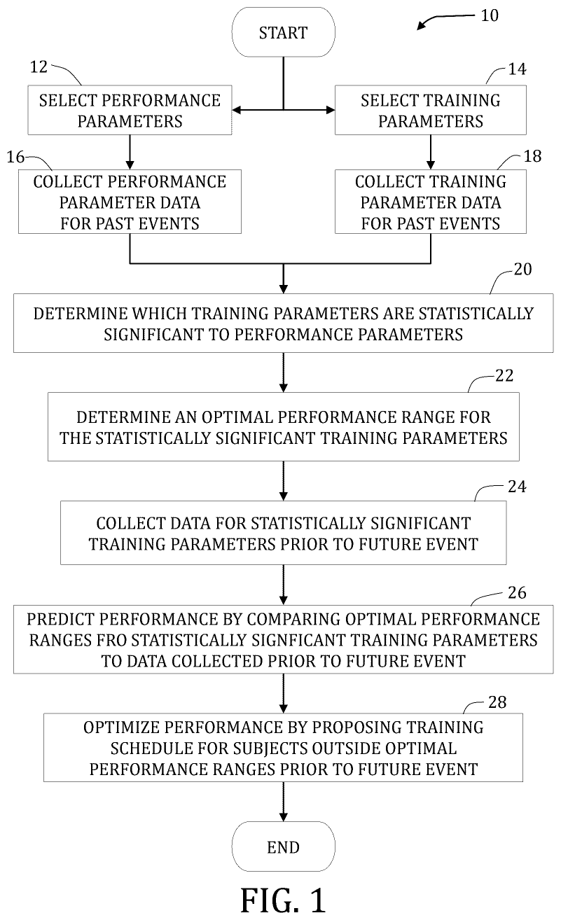

Referring to FIG. 1, a flowchart illustrating a process 10 is depicted for predicting performance, optimizing performance, or both for a future event according to one embodiment of the present invention. For purposes of the present invention, an “event” is any type of physical and/or mental activity involving skill in which a level of performance may be objectively or subjectively quantified. The event may be related to an individual sport (such as tennis, golf, cycling, running, etc.) or a team sport (such as football, basketball, baseball, lacrosse, etc.). The event may be a singular game for the particular sport or a collection of games (such as a tournament). For example, a golf tournament may include four rounds of 18 holes, each round commenced and completed on a separate day. Each round or day may be considered a separate event or the tournament, as a whole, may be considered an event, depending on a desired analysis. An event may also be related to countless other types of physical and/or mental activities not typically considered to be a sport, such as a military activity (e.g., a military mission or operation), artistic performance (such as dance, musical and theatrical performances), or largely cognitive activities (such as a chess match or a standardized examination). In some embodiments, the event may be primarily a physical activity or considered more of a physical activity than a mental activity.

For purposes of the present invention, a “past event” refers to an event in which data is collected prior to the “future event.” In other words, the term “past” is used to refer to events occurring prior to the future event for which performance is being predicted, optimized, or both. In some embodiments, past events are performed by the same individuals that will perform in the future event. That is, for individual sports, the same athlete is assessed in both past and future events; for team sports, the same group of athletes is assessed in both past and future events. According to some embodiments, the past events may be substantially similar to the future event. For example, if the future event is a basketball game, then a substantially similar past event may be a previously played basketball game, a currently played basketball game, a scrimmage, a practice, or combination thereof. If the future event is a football game, then past events may include previous football game(s), preferably by the same team members that will be competing in the future event. If the future event is a tennis match, then the past events may include a previous tennis match or matches against an opponent that is the same or similar to the opponent that is competing in the future event.

According to step 12 of process 10, one or more performance parameters for the future event are selected. Selection of performance parameters may be based on ability to be indicative of or relatedness to physical performance for the future event. For example, if the future event is a singles tennis match, then suitable performance parameters may include individual metrics, which may include an ultimate outcome of the match (i.e., win vs. loss), personal statistics that are conventionally recorded kept during a tennis match and that are indicative of performance (such as a number of aces, a number of unforced errors, a number of sets won, etc.), or a self-evaluation (for example, feeling tired, sore, mental fog, headache, etc.). If the future event is a team event, such as a basketball game, then suitable performance parameters may include individual metrics (including those provided above), team-based metrics, or both. Exemplary team-based metrics for the basketball game may include an ultimate outcome of the game (i.e., win vs. loss), team statistics that are conventionally recorded during the basketball game and that are indicative of team performance (such as points scored, points scored by opponent, team fouls, team assists, team rebounds, etc.), or team evaluation (for example, degree of communication, team unity, etc.). Individual metrics for the basketball game may be similar to the team metrics, but kept at an individual level (such as the individual's points scored, assists, rebounds, etc.). For team-based events, evaluation of performance parameters comprising both team-based and individual metrics is preferably selected.

In addition to the selection of performance parameters based on the type of future event being predicted and/or optimized, training parameters are selected to be tracked in step 14. Selection of training parameters may be based, in part, on a belief that the selected training parameters may be correlated or related to the performance parameters selected in step 12. According to certain embodiments, selection of the training parameters may be based, at least in part, on the type of data acquired or tracked by the conventional fitness tracker available to or used by the individual or team. Exemplary training parameters may include physiological data (such as heart rate, heart rate variability, respiration rate, blood oximetry, etc.) and activity-related data (such as a number of direction changes, distance covered, average speed, explosiveness, etc.). For purpose of the present invention, activity-related data may also include behavioral-related data (such as an amount of sleep, number of meals, fluid intake, overall soreness, number of sore areas, etc.). Some embodiments may include training parameters, such as caloric intake, that may be derived from combinations of multiple physiological parameters and/or activity-related parameters.

Referring to steps 16 and 18, data is collected or otherwise acquired with respect to the selected performance parameters of step 12 and the selected training parameters of step 14 for past events. For the performance parameters, data may include individual metrics, team metrics, or both, which are described above. For the training parameters, data may typically be individual-based data, such as those described above. When individual-based data for the training parameters is being correlated to team-based metrics, then a mean, a median, a weighted-average, or other statistical sampling of the individual-based data may be used for the particular training parameter. For example, if the team-based metric is whether the team won or lost and the training parameter is an amount of sleep, then the individual-based data for each team member (or each team member that participated in the past events or the future event) may be converted to team-based data (e.g., summed or averaged) for the training parameter to be correlated with the team-based data for the performance parameter for the past event.

It would be readily appreciated by those of ordinary skill in the art having the benefit of the disclosure made herein that the more related past events are to the future event, the better the predictive quality may be for the system (so long as a sufficient sample size of past events has been collected). Thus, according to certain embodiments, each past event may be assigned a characteristic indicative of the past event and loaded to a database of past events with the characteristics being identified for each of the past events. Once a significant sample size of data for past events is loaded to the database with the characteristic, a user may select certain past events from the database for correlation to a future event by narrowing the past events to those having a characteristic similar to the future event. For example, if the future event is a basketball game against a high-quality opponent as compared to a weaker opponent, then a user may select other past events (or previous games) by the same team against similar high-quality opponents to be used as past events instead of all evaluating against all past events (or all basketball games that the team has competed in). Characteristics that may be used to organize and select past events for correlation to the future event are numerous and may include, for example, the time of day in which event takes place, home game versus road game, time of travel to event, proximity in time to other events, weather during event (e.g., temperature, rain, sun, snow, humidity, etc.), expected duration of the event, and so forth.

For team sports, past events may be characterized by the players involved during the event. For example, if a star player was injured or otherwise not available during a past event, then that past event may be removed from the analysis if the star player is expected to participate in the future event. Similarly, a new individual to a team may be initially correlated with past events in which the new individual did not participate, and then data collected during the future event in which the new individual did participate may be loaded to the database for validating the initial correlation.

It would be readily understood by the skilled artisan that once the future event has concluded, data collected with respect to the future event may be used as a “past event.” In this way, the predictive quality of the data and the system may be continually updated and improved.

With respect to step 16 and data being collected for the selected performance parameters for past events, the data may be either objectively quantified or subjectively quantified. For example, if the event is a basketball game, team and/or individual performance parameters such as win vs. loss, number of assists, number of points, points by opponent, etc. are able to be objectively quantified by collecting the data during the past event. However, particularly for individual sports or individual data within a team sport, subjective quantification may be used such as “rate your performance today on a scale of 1-10” using a subject survey or third-party ratings (e.g., coach ratings) after the past event is completed.

Similarly, with respect to step 18 and data being collected for the selected training parameters for past events, the data is preferably objectively quantified when possible and subjectively quantified when needed. Most of the training parameter data should be able to be objectively quantified using commercially-available fitness trackers. On the other hand, other training parameter data such as number of meals eaten and fluid intake may need to be objectively quantified data using a subject survey. The subject survey may also be used to collect the data for subjectively quantified training parameter data such as total soreness (e.g., “rate how sore you are on a scale of 1-10”), number of sore areas, emotional stress, etc.

With continuing reference to step 18, and according to some embodiments of the present invention, training parameter data may be grouped into a plurality of time periods based on when the data was collected as compared to the past event to which the training parameter data corresponds. For example, training parameter data (such as change of direction, max acceleration, distance ran, etc.) may be collected during training sessions for a few days prior to the future event, which may then be grouped by the number of days in which the training session was held before the corresponding past event (e.g., one data point for the number of times the individual changed directions during a training session four days before the event, another data point for the number of times the athlete changed directions during a training session three days before the event, and so forth). Data for certain training parameters, particularly physiological parameters (such as heart rate and heart rate variability), may also be collected a plurality of times during a day (e.g., one data point for heart rate when the athlete woke up on the morning four days before the event, one data point for heart rate before the athlete went to bed four nights before the event, . . . , and one data point for heart rate on the morning of the event).

Once training parameter data and corresponding performance parameter data are collected in steps 16 and 18 for a sufficient number of past events, step 20 of the process 10 determines which of the selected training parameters of step 14 are statistically significant to the selected performance parameters of step 12. When data for training parameters is grouped into a plurality of time periods, step 20 may further include determining the statistically significance of training parameters for each of the plurality of time periods. For purposes of the present invention, one parameter is “statistically significant” to another parameter when the parameters pass a statistically significant test as known in the art to a certain predetermined confidence level. For example, according to certain embodiments, if a correlation between particular training parameter and a performance parameter has a p-value of 10% or less, then the training parameter may be considered to be statistically significant. In other words, in situations in which there is 10% or less chance that the relationship between the training parameter and the performance parameter was the result of chance, the training parameter may be considered to be statistically significant to the performance parameter. On the other hand, if the p-value is greater than 10%, then the parameter may be considered to be statistically insignificant. As data is collected for more and more past events, the predetermined confidence level (for example, the p-value) may be changed to require a greater confidence level (such as a p-value of 5% or less). In addition to p-values, the statistically significance may be determined by other statistics tests and techniques such as genetic algorithms, lass net models, etc.

The statistically significant training parameters identified in step 20 may be used in a variety of ways in relationship to performance at future events. For example, referring to steps 22-26, performance for a future event may be predicted to modify strategy for the future event. In this regard, in step 22, an optimal performance range for a training parameter may be determined for by comparing data associated with the particular training parameter for each past event to the performance parameter data of the same past event. Again, when data for the training parameter is grouped into a plurality of different time periods, then step 22 may further include determining an optimal performance range for each time period of the plurality. The optimal performance range may be preferably determined by a statistical distribution type analysis (such as shown in a bell curve, z-table, etc.). Alternately, a prediction model may be used where potential values for the data are used in the models to determine how the performance parameter would change in response to the training parameter data. An optimal performance range may then be chosen based on the prediction model results. In step 24, data is collected for the particular training parameter prior to the future event. Then, in step 26, performance may be predicted by comparing the training parameter data from step 24 to the optimal performance range for the parameter determined in step 22.

As would be understood, the ability to predict performance may result in significant advantages. For example, as further explained below, it has been determined that heart rate variability is often a statistically significant training parameter when compared to performance parameters of different types of events. Thus, assuming heart rate variability is found to be statistically significant training parameter for an individual basketball player or a basketball team in step 20 and an optimal performance range for heart rate variability for the individual or team is determined in step 22, then a coach may decide to sit or otherwise adjust playing times for the individual basketball player during the future event if data collected during step 24 shows the individual basketball player's heart rate variability to be outside of the optimal performance range. In other words, the coach is able to recognize, ahead of time, when there is a statistically good chance that a player will have a less than optimal performance during a particular future event. Accordingly, the coach may use this information to determine whether the player should receive more or less playing time than normal during the game.

Similarly, referring to step 28, the optimal performance ranges determined in step 22 and training parameter data collected in step 24 may also be used to optimize performance during a future event by detecting that a player or team is outside the particular optimal performance range that was determined in step 26. And so, continuing with the heart rate variability example above, assuming in step 20 that heart rate variability was determined to be statistically significant two days before a game, then in step 26, it is then determined that a player's heart rate variability is outside the individual's and/or team's optimal performance range three-four days before the game, then in step 28, expected performance may be improved by proposing a training schedule that is designed to get the athlete's heart rate variability to conform to the optimal performance range.

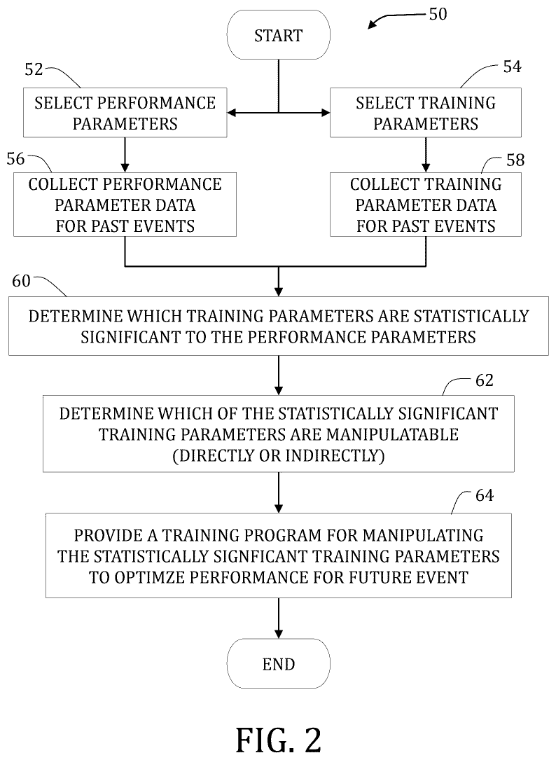

Referring now to FIG. 2, a flowchart illustrating a process 50 for optimizing performance according to another embodiment of the present invention is described. Similar to the process 10 of FIG. 1, process 50 may include determining which training parameters are statistically significant to the performance parameters for a particular type of event by selecting performance parameters in step 52, selecting training parameters in step 54, collecting performance parameter data for past events in step 56, collecting training parameter data for past events in step 58, and determining which training parameters are statistically significant to the performance parameters in step 60. To optimize performance, process 50 further includes determining which of the statistically significant training parameters are manipulatable in step 62. Directly manipulatable parameters may include, for example, a number of reps (such as a number of changes in direction during a training session); indirectly manipulatable parameters may include, for example, results from level of training (e.g., overall soreness of an athlete may be found to be indirectly changed by adjusting a number of changes in directing during a training session). Thus, step 62 may include determining which statistically significant training parameters may be changed directly and which statistically significant training parameters must be changed indirectly or are otherwise difficult to change directly.

According to certain embodiments, determining the manipulatable parameters in step 62 includes comparing the statistically significant training parameters to the other training parameters to determine whether and which of the other training parameters may be statistically significant to a particular statistically significant training parameter. For example, supposing heart rate variability is found to be a statistically significant training parameter to performance in step 60, then while heart rate variability may difficult to manipulate directly, step 62 enables the identifying of training parameters that are best suited to manipulate the heart rate variability indirectly based on relatedness by statistical significance. That is, by determining which training parameters are statistically significant to heart rate variability according to a pre-determined confidence level. Accordingly, step 62 provides the ability to determine which training parameters may be directly manipulated, that are not necessarily statistically significant to performance parameters, but that may be used to indirectly manipulate the training parameter that is unable to be manipulated directly.

As should be understood, certain training parameters could be found to be both a statistically significant training parameter for a particular performance parameter as well as a statistically significant training variable to another statistically significant training parameter.

Once it is determined which training parameters are statistically significant to performance parameters in step 60 and which training parameters may be used to manipulate the statistically significant training parameters as determined in step 62, then step 64 of process 50 includes providing a training program for manipulating the statistically significant training parameters prior to the future event so as to optimize performance parameters during the future event.

In certain embodiments, process 50 further includes developing an optimal performance range for the statistically significant training parameters in a manner that may be similar to step 22 of process 10. According to this embodiment, the training program of step 64 includes manipulating the statistically significant training parameters so that the parameters conform to the optimal performance range.

In an alternate embodiment of process 50, particularly when it is already known which training parameters should be statistically significant for a particular event, step 60 of process 50 may include selection of training parameters believed to be indicative of performance instead of actually performing the statistically significant test. Thus, according to this embodiment, process 50 may include determining the manipulatable parameters in step 62 for the training parameters selected without necessarily requiring data to be collected regarding the performance parameters of past events. In other words, a modified version of process 50 may be used to determine the statistically significant training variables of pre-selected training parameters that are already understood or believed to be statistically significant to performance for an event.

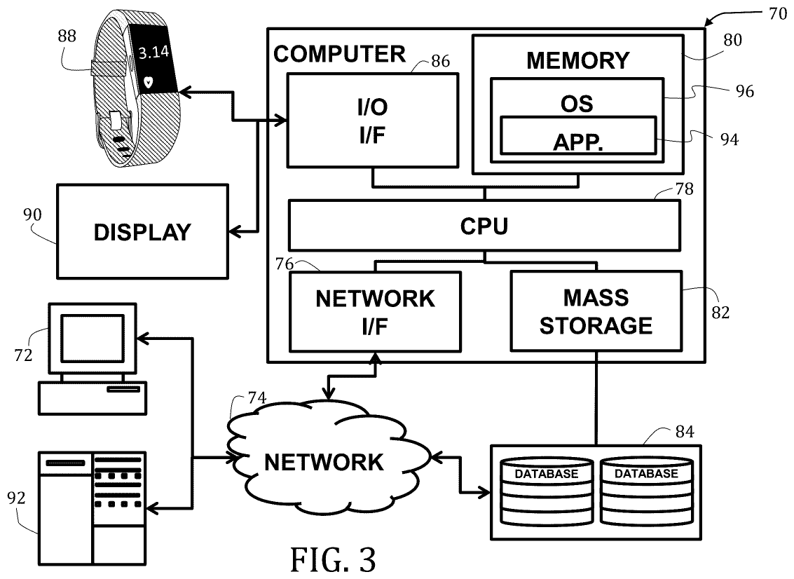

According to another aspect of the invention, a system 70 for performing the process steps described above is provided. In some embodiments, the system 70 may comprise a computing system; according to other embodiments, the system 70 may include software or computing program. The details of a computing system 70 suitable for performing methods according to the various embodiments of the present invention is described with reference to FIG. 3. The illustrative computing system 70 may be considered to represent any type of computer, computer system, computing system, server, disk array, or programmable device such as multi-user computers, single-user computers, handheld devices, networked devices, or embedded devices, etc. The computing system 70 may be implemented with one or more networked computers 72 using one or more networks 74, e.g., in a cluster or other distributed computing system through a network interface 76 (illustrated as “NETWORK I/F”). The computing system 70 will be referred to as “computer” for brevity's sake, although it should be appreciated that the term “computing system” may also include other suitable programmable electronic devices consistent with embodiments of the invention.

The computer 70 typically includes at least one processing unit 78 (illustrated as “CPU”) coupled to a memory 80 along with several different types of peripheral devices, e.g., a mass storage device 82 with one or more databases 84, an input/output interface 86 (illustrated as “I/O I/F”) coupled to a user input (illustrated a fitness tracker 88) and a display 90, and the Network I/F 76. The memory 80 may include dynamic random access memory (“DRAM”), static random access memory (“SRAM”), non-volatile random access memory (“NVRAM”), persistent memory, flash memory, at least one hard disk drive, and/or another digital storage medium. The mass storage device 82 is typically at least one hard disk drive and may be located externally to the computer 70, such as in a separate enclosure or in one or more networked computers 72, one or more networked storage devices (including, for example, a tape or optical drive), and/or one or more other networked devices (including, for example, a server 92).

The CPU 78 may be, in various embodiments, a single-thread, multi-threaded, multi-core, and/or multi-element processing unit (not shown) as is well known in the art. In alternative embodiments, the computer 70 may include a plurality of processing units that may include single-thread processing units, multi-threaded processing units, multi-core processing units, multi-element processing units, and/or combinations thereof as is well known in the art. Similarly, the memory 80 may include one or more levels of data, instruction, and/or combination caches, with caches serving the individual processing unit or multiple processing units (not shown) as is well known in the art.

The memory 80 of the computer 70 may include one or more applications 94 (illustrated as “APP.”), or other software program, which are configured to execute in combination with the Operating System 96 (illustrated as “OS”) and automatically perform tasks necessary for performing embodiments of the present invention, with or without accessing further information or data from the database(s) 84 of the mass storage device 82. The memory 80, APP. 94, and so forth may, by way of the user interface 86, allow a user to select the various performance and training parameters being tracked for past and future events and upload the collected data for the parameters preferably directly from commercially-available fitness monitors 88. The memory 80, APP. 94, or other software program may then use the collected data to perform the statistical analysis process steps as described above, including determining the statistically significant training parameters, the statistically training variables, optimal ranges for the statistically significant training parameters, and generating training programs for optimizing performance.

Those skilled in the art will recognize that the environment illustrated in FIG. 3 is not intended to limit the present invention. Indeed, those skilled in the art will recognize that other alternative hardware and/or software environments may be used without departing from the scope of the invention.

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

### Examples

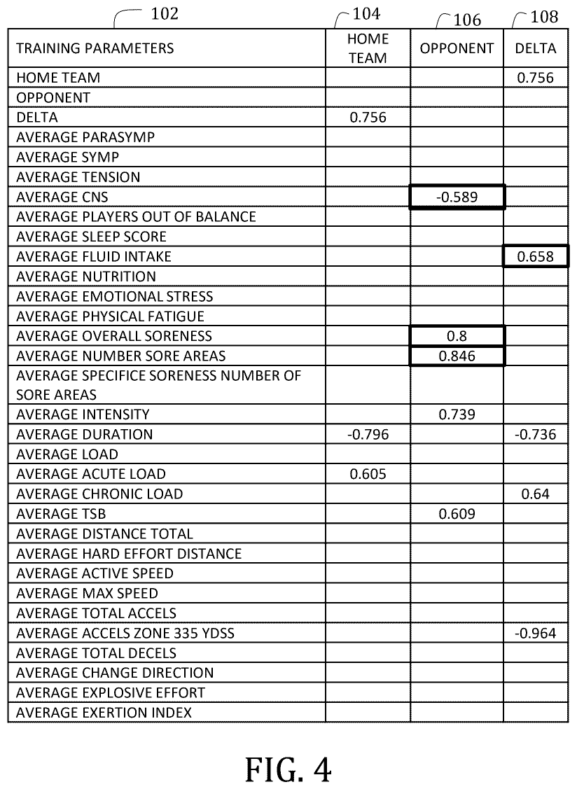

Referring to FIGS. 4-8, an example of the processes described above and according to embodiments of the present invention is provided using sample data from a men's lacrosse team referred to in the example as “HOME TEAM.” Referring first to FIG. 4, the selected training parameters are shown in the left-hand column 100. In this instance, because the event is a team-based event, the collected data for the individual-based training parameters were averaged to arrive at team-based data. The performance parameters in this example were goals scored by HOME TEAM, goals scored by the opponent, and the goal differential. The second column 102 identifies which training parameters were found to be statistically significant to the number of goals scored by HOME TEAM for the past events, the third column 104 identifies which training parameters were found to be statistically significant to the goals scored by the opponent, and the last column 106 identifies which training parameters were found to be statistically significant to the goal differential. Positive values indicate statistically significant training parameters that increased the corresponding performance parameter when the value of the training parameter increased (e.g., more goals were scored by HOME TEAM when “Average Acute Load” scores increased) and the negative values indicate statistically significant training parameters that had increased the corresponding performance parameter when the value of the training parameter decreased (e.g., more goals were scored by HOME TEAM when “Average Duration” scores decreased).

As shown in FIG. 4, two of the statistically significant training parameters for the performance parameter of goals scored by HOME TEAM's opponent as highlighted in column 106 were found to be “Average Overall Soreness” and “Average Number Sore Areas.” These training parameters are difficult to manipulate directly. Thus, referring to FIG. 4, certain training parameters identified in column 110 were used to determine which of the training parameters of column 110 were statistically significant training variables for “Average Overall Soreness” and “Average Number Sore Areas.” Referring to column 112, the parameters of “Average Sleep Score,” “Average Emotional Stress,” “Average Number Sore Areas,” “Average Specific Soreness Number Sore Areas,” “Average Change Direction,” and “Average Explosive Effort” were determined to be statistically significant training variables for “Average Overall Soreness.” While it is expected that this information can be used in a variety of ways, for purposes of the present example it is noted that, of these statistically significant training variables identified in column 112, “Average Sleep Score,” “Average Change Direction,” and “Average Explosive Effort” are able to be manipulated directly. Thus, this information can be utilized to implement a training program for the team to indirectly manipulate the “Average Overall Soreness” to optimize performance by directly manipulating one or more of the statistically significant training variables. A similar analysis can be used with respect to the statistically significant training variables for the “Average Number Sore Areas” as identified in column 114.

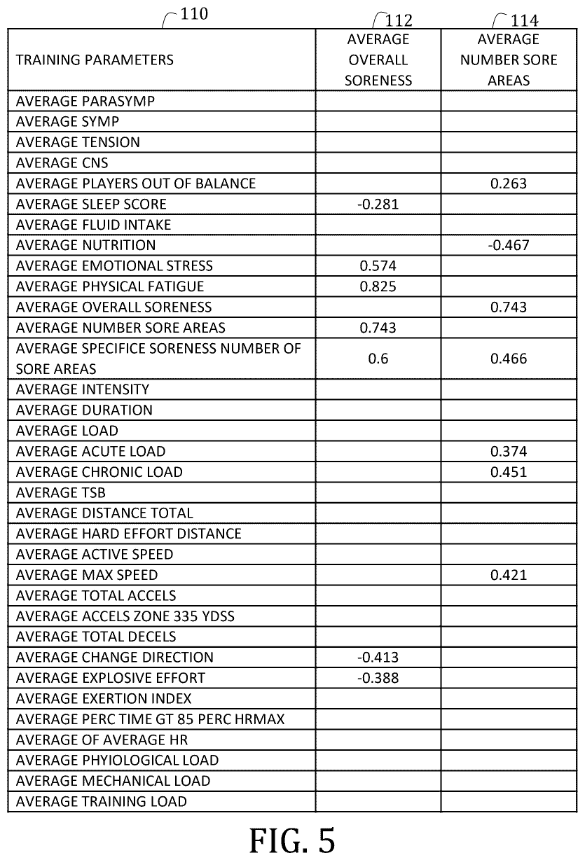

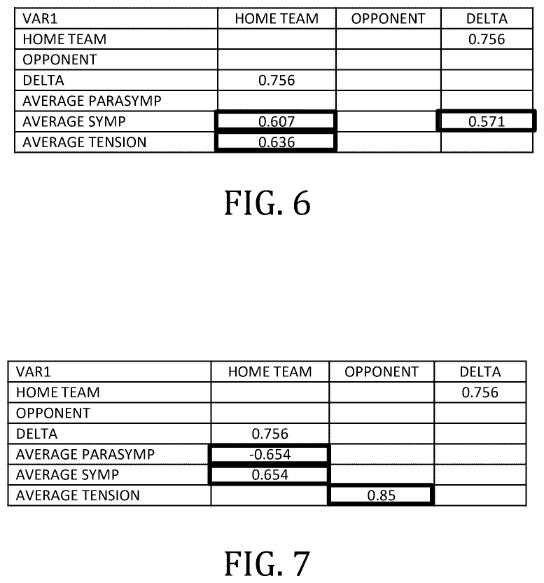

It is noted that the training parameter data used in the example of FIGS. 4 and 5 was for the day of the past event to which the data corresponded. Referring to the tables of FIGS. 5 and 6, statistically significant training parameters were also investigated for two days prior to the past events (FIG. 6) and five days prior to the past events (FIG. 6). As shown in these tables, tension and heart rate variability (either parasympathetic or sympathetic values for heart rate variability) were determined to be statistically significant training parameters. In particular, for two days prior to an event, it was found that the higher the average sympathetic score for the team, the more points scored on game day and the greater the point differential in the favor of HOME TEAM. For five days prior to an event, it was found that lower parasympathetic scores and higher sympathetic scores increased the number of goals scored by HOME TEAM.

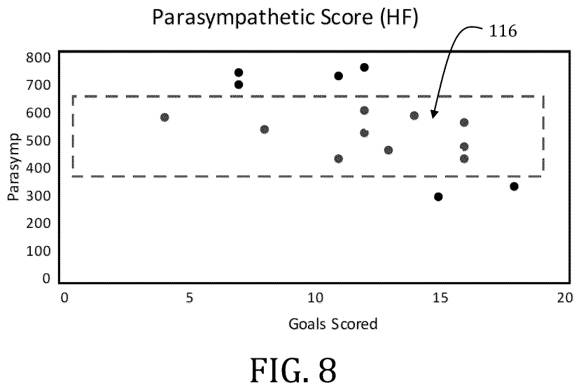

Referring again to FIG. 7 and as noted above, it was found in this example that lower parasympathetic scores increased performance for HOME TEAM. Thus, referring to FIG. 8, a distribution analysis was performed on the statistically significant training parameter of parasympathetic scores five days before a game to determine an optimal performance range as identified in box 116. It is noted that the finding that lower parasympathetic scores within a resting range (e.g., taken when the individual wakes up in the morning) of about 300 ms² to about 650 ms² highlights the utility of the present invention in that lower parasympathetic scores are typically believed to decrease performance instead of increase performance. Thus, at least for the team represented by FIGS. 7 and 8, the present system discovered that their performance is improved in a manner that is inconsistent or counterintuitive with respect to traditional thinking.

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

1. A system for optimizing human physiological performance with respect to a set of physiological and activity-related parameters related to a particular event, the system comprising:
a display configured to operate as a user interface capable of receiving a user input and displaying an output to the user,
a memory, storing one or more processor-readable instructions, and
a processor, capable of receiving user inputs and displaying user outputs on the display based on the one or more processor-readable instructions, which are further configured to cause the system to:
receive a selection of one or more individual performance parameters for a future instance of the particular event from the user interface, the individual performance parameters indicative of a desired physical performance of an individual for the particular event;
receive a selection of one or more team performance parameters for a future instance of the particular event from the user interface, the team performance parameters indicative of a physical performance of a team for the particular event;
collect data related to the individual performance parameters and the team performance parameters from past occurrences of the particular event;
identify a subset of training parameters, from within a collection of training parameter data associated with past occurrences of the particular event or related events, that exhibit a statistically significant correlation with the selected individual performance parameters and the selected team performance parameters; and
provide a training program, accessible on the user interface, listing each of the optimal training parameters and recommending a training routine for optimizing each of the individual performance parameters and the team performance parameters.

2. The system of claim 1, further configured to:
group the collection of training parameter data associated with past occurrences of the particular event or related events into a plurality of time periods based on how long the data was collected prior to the future instance of the particular event, and
determine which of the selected training parameters are statistically significant training parameters for each of the plurality of time periods.

3. The system of claim 1, further comprising a wearable fitness monitor for collecting data for one or more of the selected training parameters.

4. The system of claim 1, wherein a statistically significant correlation between the training parameters and the selected individual performance parameters and the selected team performance parameters is determined based on a predetermined confidence level.

5. The system of claim 4, wherein the confidence level is 90%.

6. The system of claim 4, wherein the required confidence level for a statistically significant correlation between the training parameters and the selected individual performance parameters increases with increased numbers of recorded events.

7. The system of claim 6, wherein the required confidence level for a statistically significant correlation between the training parameters and the selected individual performance parameters is based on one or more genetic algorithms or lasso net models.

8. A method for optimizing human physiological performance with respect to a set of physiological and activity-related parameters related to a particular event, the method comprising:
receiving, via a user interface, a selection of one or more individual performance parameters for an instance of the particular event, the individual performance parameters indicative of a desired physical performance of an individual for the particular event;
receiving, via the user interface, a selection of one or more team performance parameters for an instance of the particular event, the team performance parameters indicative of a physical performance of a team for the particular event;
collecting data related to the individual performance parameters and the team performance parameters from past occurrences of the particular event;
accessing training parameter data associated with the past occurrences of the particular event;
identifying, from the training parameter data, a subset of training parameters that exhibit a statistically significant correlation with the individual performance parameters and the team performance parameters;
generating a training program that lists each of the identified training parameters and recommends a training routine for optimizing the individual performance parameters and the team performance parameters; and
providing the training program to a user via the user interface.

9. The method of claim 8, further comprising predicting, based on the identified subset of training parameters, a future value of at least one of the individual performance parameters for a future instance of the particular event.

10. The method of claim 9, further comprising comparing the predicted value of the individual performance parameter to an individual performance parameter goal value provided by the user via the user interface.

11. The method of claim 10, further comprising modifying the training program in response to a difference between the predicted value and the individual performance parameter goal.

12. The method of claim 9, further comprising updating the predicted future value based on new training parameter data collected after the training program is generated.

13. The method of claim 8, wherein a statistically significant correlation between the training parameters and the selected individual performance parameters and the selected team performance parameters is determined based on a predetermined confidence level.

14. The method of claim 13, wherein the confidence level is 90%.

15. The method of claim 13, wherein the required confidence level for a statistically significant correlation between the training parameters and the selected individual performance parameters increases with increased numbers of recorded events.

16. The method of claim 15, wherein the required confidence level for a statistically significant correlation between the training parameters and the selected individual performance parameters is based on one or more genetic algorithms or lasso net models.

17. A system for facilitating improvement of human physiological performance relative to a particular event, the system comprising:
a display configured as a user interface for receiving user input and presenting information to a user;
a memory storing one or more processor-executable instructions; and
a processor operably coupled to the display and the memory, the processor configured to execute the instructions to:
obtain, via the user interface, input identifying one or more individual performance parameters associated with a future instance of the particular event, the individual performance parameters indicative of a desired outcome for an individual participant in the event;
obtain, via the user interface, input identifying one or more team performance parameters associated with the future instance of the particular event, the team performance parameters indicative of a desired outcome for a group of participants in the event;
access stored data representing historical instances of the particular event or related events, the historical data comprising training parameter data and corresponding performance outcomes;
determine a set of training parameters, from the historical training parameter data, that exhibit a predictive relationship with the identified individual performance parameters and team performance parameters, based on a statistical analysis of the historical data;
generate and display, via the user interface, a training program including training recommendations corresponding to the determined training parameters, the training recommendations being configured to guide the individual in achieving the identified individual performance parameters and team performance parameters.

18. The system of claim 17, wherein the predictive relationship is based on a statistically significant correlation between the training parameters and the selected individual performance parameters and the selected team performance parameters.

19. The system of claim 18, wherein the statistically significant correlation is determined based on a predetermined confidence level.

20. The system of claim 19, wherein the required confidence level for a statistically significant correlation between the training parameters and the selected individual performance parameters increases with increased numbers of recorded events.
